(19) United States
(12) Patent Application Publication
(10) Pub. No.: US 2018/0298308 A1
(43) Pub. Date: Oct. 18, 2018

(54) STORAGE SOLUTION FOR OPHTHALMIC LENS AND OPHTHALMIC PRODUCT USING THE SAME

(71) Applicant: HON HAI PRECISION INDUSTRY CO., LTD., New Taipei (TW)

(72) Inventor: HSIU-WEN CHIEN, New Taipei (TW)

(21) Appl. No.: 15/498,637

(22) Filed: Apr. 27, 2017

(30) Foreign Application Priority Data
Apr. 18, 2017 (TW) 106112906

(51) Int. Cl. C11D 3/00; A61L 12/08; A61L 12/14; C11D 1/90; C11D 3/04; C11D 3/30; C11D 3/20; A45C 11/00; B65D 81/22

(52) U.S. Cl. CPC C11D 3/0078; A61L 12/086; A61L 12/143; C11D 1/90; B65D 81/22; C11D 3/30; C11D 3/2065; A45C 11/005; C11D 3/046

(57) ABSTRACT

A storage solution for an ophthalmic lens comprises an osmotic pressure adjusting agent, sodium chloride, and deionized water. The osmotic pressure adjusting agent is selected from a group consisting of betaine, L-carnitine, and erythritol, or any combination thereof. The storage solution has an osmotic pressure of about 280 mOsm/kg to about 320 mOsm/kg. The disclosure also provides an ophthalmic product using the storage solution.

### FIELD

The subject matter herein generally relates to a storage solution, and more particularly, to a storage solution for an ophthalmic lens and an ophthalmic product using the storage solution.

### BACKGROUND

Ophthalmic lenses are commonly worn by users to correct vision, or for cosmetic or therapeutic reasons. Usually, the ophthalmic lens is immersed in a storage solution to keep hydrated. The storage solution comprises sodium chloride to adjust an osmotic pressure of the storage solution to provide the user with a wear comfort. However, the storage solution may need a high content of sodium chloride for a desired osmotic pressure, which may destroy an ionic balance in cells of eyes and thus reduce an activity of the cells.

### BRIEF DESCRIPTION OF THE DRAWINGS

Implementations of the present technology will now be described, by way of example only, with reference to the attached figures.

FIG. 1 is a cross-sectional view of an exemplary embodiment of an ophthalmic product.

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The term “comprising” when utilized, means “including, but not necessarily limited to”; it specifically indicates open-ended inclusion or membership in the so-described combination, group, series and the like.

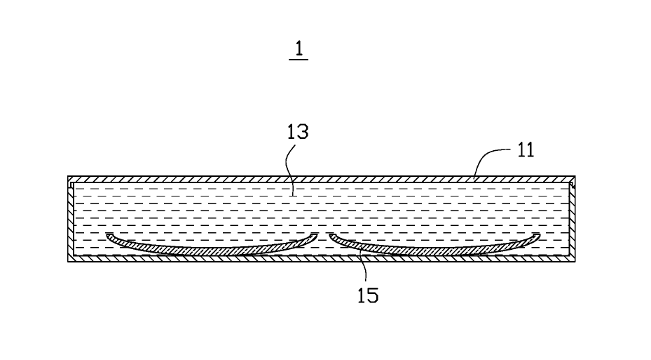

FIG. 1 illustrates an exemplary embodiment of a storage solution 13 for ophthalmic lens 15. The storage solution 13 comprises an osmotic pressure adjusting agent, sodium chloride, and deionized water. The osmotic pressure adjusting agent and sodium chloride are dissolved in the deionized water. The osmotic pressure adjusting agent can be selected from a group consisting of betaine, L-carnitine, and erythritol, or any combination thereof.

The storage solution 13 has an osmotic pressure of about 280 mOsm/kg to about 320 mOsm/kg at room temperature. In at least one exemplary embodiment, the storage solution 13 has an osmotic pressure of about 285 mOsm/kg to about 310 mOsm/kg.

The osmotic pressure adjusting agent has a mass percentage of about 0.005% to about 0.25% of a total mass of the storage solution 13. The sodium chloride has a mass percentage of about 0.35% to about 0.9% of the total mass of the storage solution 13. In at least one exemplary embodiment, the sodium chloride has a mass percentage of about 0.5% to about 0.85% of the total mass of the storage solution 13.

The storage solution 13 has a potential of hydrogen (PH) value of about 7.1 to about 7.4. In at least one exemplary embodiment, when the storage solution 13 has the PH value less than 7.1 or greater than 7.4, the storage solution 13 can further comprise a potential of hydrogen adjusting agent to adjust the pH value of the storage solution 13. The potential of hydrogen adjusting agent may be selected from a group consisting of boracic acid, sodium borate, sodium carbonate, sodium bicarbonate, edetic acid (EDTA), disodium ethylenediaminetetraacetate (disodium EDTA), monopotassium phosphate, monosodium phosphate, and sodium hydrogen phosphate, or any combination thereof.

The storage solution 13 is made by mixing the osmotic pressure adjusting agent, the sodium chloride, and the deionized water, and dissolving the osmotic pressure adjusting agent and the sodium chloride in the deionized water.

### EXAMPLE 1

The storage solution comprises betaine, sodium chloride, and deionized water. The betaine had a mass percentage of 0.018% of a total mass of the storage solution. The sodium chloride had a mass percentage of 0.81% of the total mass of the storage solution. The deionized water had a mass percentage of 99.172% of the total mass of the storage solution. The storage solution had an osmotic pressure of 289.2 mOsm/kg and a PH value of 7.38.

### EXAMPLE 2

The storage solution comprises betaine, L-carnitine, sodium chloride, and deionized water. The betaine had a mass percentage of 0.007% of a total mass of the storage solution. The L-carnitine had a mass percentage of 0.011% of the total mass of the storage solution. The sodium chloride had a mass percentage of 0.56% of the total mass of the storage solution. The deionized water had a mass percentage of 99.422% of the total mass of the storage solution. The storage solution had an osmotic pressure of 291.5 mOsm/kg and a PH value of 7.4.

### EXAMPLE 3

The storage solution comprises erythritol, L-carnitine, sodium chloride, and deionized water. The erythritol had a mass percentage of 0.012% of a total mass of the storage solution. The L-carnitine had a mass percentage of 0.016% of the total mass of the storage solution. The sodium chloride had a mass percentage of 0.54% of the total mass of the storage solution. The deionized water had a mass percentage of 99.432% of the total mass of the storage solution. The storage solution had an osmotic pressure of 290.5 mOsm/kg and a PH value of 7.38.

### EXAMPLE 4

The storage solution comprises betaine, erythritol, L-carnitine, sodium chloride, and deionized water. The betaine had a mass percentage of 0.008% of a total mass of the storage solution. The erythritol had a mass percentage of 0.01% of the total mass of the storage solution. The L-carnitine had a mass percentage of 0.007% of the total mass of the storage solution. The sodium chloride had a mass percentage of 0.61% of the total mass of the storage solution. The deionized water had a mass percentage of 99.365% of the total mass of the storage solution. The storage solution had an osmotic pressure of 290.3 mOsm/kg and a PH value of 7.4.

### EXAMPLE 5

The storage solution comprises L-carnitine, sodium chloride, and deionized water. The L-carnitine had a mass percentage of 0.019% of a total mass of the storage solution. The sodium chloride had a mass percentage of 0.71% of the total mass of the storage solution. The deionized water had a mass percentage of 99.271% of the total mass of the storage solution. The storage solution had an osmotic pressure of 285.3 mOsm/kg and a PH value of 7.3.

### EXAMPLE 6

The storage solution comprises erythritol, sodium chloride, and deionized water. The erythritol had a mass percentage of 0.018% of a total mass of the storage solution. The sodium chloride had a mass percentage of 0.75% of the total mass of the storage solution. The deionized water had a mass percentage of 99.232% of the total mass of the storage solution. The storage solution had an osmotic pressure of 289 mOsm/kg and a PH value of 7.35.

### EXAMPLE 7

The storage solution comprises betaine, erythritol, sodium chloride, and deionized water. The betaine had a mass percentage of 0.012% of a total mass of the storage solution. The erythritol had a mass percentage of 0.008% of the total mass of the storage solution. The sodium chloride had a mass percentage of 0.79% of the total mass of the storage solution. The deionized water had a mass percentage of 99.19% of the total mass of the storage solution. The storage solution had an osmotic pressure of 290 mOsm/kg and a PH value of 7.4.

Referring to FIG. 1, an ophthalmic product 1 comprises a packaging housing 11, the storage solution 13, and at least one ophthalmic lens 15. The storage solution 13 and the at least one ophthalmic lens 15 are received in the packaging housing 11. Each ophthalmic lens 15 is immersed in the storage solution 13.

The osmotic pressure adjusting agent can adjust the osmotic pressure of the storage solution 13 to allow the user to feel comfortable when the ophthalmic lens 15 is worn by the user. Furthermore, the osmotic pressure adjusting agent is not an ionic type adjusting agent, thereby preventing an ionic balance in cells of eyes being destroyed.

It is to be understood, even though information and advantages of the present embodiments have been set forth in the foregoing description, together with details of the structures and functions of the present embodiments, the disclosure is illustrative only; changes may be made in detail, especially in matters of shape, size, and arrangement of parts within the principles of the present embodiments to the full extent indicated by the plain meaning of the terms in which the appended claims are expressed.

1. A storage solution for an ophthalmic lens comprising:
erythritol;
sodium chloride; and
deionized water;
wherein the storage solution has an osmotic pressure of about 280 mOsm/kg to about 320 mOsm/kg, the erythritol has a mass percentage of 0.005% to 0.25% of a total mass of the storage solution, and the sodium chloride has a mass percentage of 0.35% to 0.9% of the total mass of the storage solution.

2. (canceled)

3. The storage solution of claim 1, wherein the storage solution has a potential of hydrogen value of 7.1 to 7.4.

4. The storage solution of claim 1, further comprising a potential of hydrogen adjusting agent.

5. The storage solution of claim 4, the potential of hydrogen adjusting agent is selected from a group consisting of boracic acid, sodium borate, sodium carbonate, sodium bicarbonate, edetic acid, disodium ethylenediaminetetraacetate, monopotassium phosphate, monosodium phosphate, and sodium hydrogen phosphate, or any combination thereof.

6. An ophthalmic product comprising:
a packaging housing;
at least one ophthalmic lens; and
a storage solution comprising:
erythritol;
sodium chloride; and
deionized water;
wherein the storage solution and the at least one ophthalmic lens are received in the packaging housing, and the storage solution has an osmotic pressure of about 280 mOsm/kg to about 320 mOsm/kg, the erythritol has a mass percentage of 0.005% to 0.25% of a total mass of the storage solution, and the sodium chloride has a mass percentage of 0.35% to 0.9% of the total mass of the storage solution.

7. (canceled)

8. The ophthalmic product of claim 6, wherein the storage solution has a potential of hydrogen value of 7.1 to 7.4.

9. The ophthalmic product of claim 6, wherein the storage solution further comprises a potential of hydrogen adjusting agent.

10. The ophthalmic product of claim 9, wherein the potential of hydrogen adjusting agent is selected from a group consisting of boracic acid, sodium borate, sodium carbonate, sodium bicarbonate, edetic acid, disodium ethylenediaminetetraacetate, monopotassium phosphate, monosodium phosphate, and sodium hydrogen phosphate, or any combination thereof.
